(19) United States
(12) Patent Application Publication
(10) Pub. No.: US 2004/0248879 A1
(43) Pub. Date: Dec. 9, 2004

(54) Salts of tricyclic inhibitors of poly(ADP-ribose) polymerases

(75) Inventors: Stacie Canan-Koch, La Jolla, CA (US); Jan-Jon Chu, Carlsbad, CA (US); Jia Liu, La Jolla, CA (US); Jean Matthews, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/811,513

(22) Filed: Mar. 29, 2004

Related U.S. Application Data
(60) Provisional application No. 60/459,433, filed on Mar. 31, 2003.

(51) Int. Cl. A61K 31/675; A61K 31/55; C07D 487/02

(52) U.S. Cl. 514/215; 540/577

(57) ABSTRACT

Pharmaceutically acceptable salts of compounds of the formula below are poly(ADP-ribosyl)transferase (PARP) inhibitors, and are useful as therapeutics in treatment of cancers and the amelioration of the effects of stroke, head trauma, and neurodegenerative disease. As cancer therapeutics, the compounds of the invention may be used, e.g., in combination with cytotoxic agents and/or radiation.

### FIELD OF THE INVENTION

The invention pertains to the salts of 8-fluoro-2-(4-methylaminomethyl-phenyl)-1,3,4,5-tetrahydro-azepino[5,4,3-cd]indol-6-one, a compound that inhibits poly(ADP-ribose) polymerases, thereby retarding the repair of damage to DNA strands, and to methods of preparing such compounds. The invention also relates the use of such compounds in pharmaceutical compositions and therapeutic treatments useful for potentiation of anti-cancer therapies and inhibition of neurotoxicity consequent to stroke, head trauma, and neurodegenerative diseases.

### BACKGROUND OF THE INVENTION

Poly(ADP-ribose) polymerases (PARPs), nuclear enzymes found in almost all eukaryotic cells, catalyze the transfer of ADP-ribose units from nicotinamide adenine dinucleotide (NAD⁺) to nuclear acceptor proteins, and are responsible for the formation of protein-bound linear and branched homo-ADP-ribose polymers. Activation of PARP and resultant formation of poly(ADP-ribose) can be induced by DNA strand breaks after exposure to chemotherapy, ionizing radiation, oxygen free radicals, or nitric oxide (NO).

Because this cellular ADP-ribose transfer process is associated with the repair of DNA strand breakage in response to DNA damage caused by radiotherapy or chemotherapy, it can contribute to the resistance that often develops to various types of cancer therapies. Consequently, inhibition of PARP may retard intracellular DNA repair and enhance the antitumor effects of cancer therapy. Indeed, in vitro and in vivo data show that many PARP inhibitors potentiate the effects of ionizing radiation or cytotoxic drugs such as DNA methylating agents. Therefore, inhibitors of the PARP enzyme are useful as cancer chemotherapeutics.

In addition, it has been shown that inhibition of PARP promotes resistance to brain injury after stroke (Endres et al., “Ischemic Brain Injury is Mediated by the Activation of Poly(ADP-Ribose) Polymerase,” J. Cerebral Blood Flow Metab. 17:1143-1151 (1997); Zhang, “PARP Inhibition Results in Substantial Neuroprotection in Cerebral Ischemia,” Cambridge Healthtech Institute's Conference on Acute Neuronal Injury: New Therapeutic Opportunities, Sep. 18-24, 1998, Las Vegas, Nev.). The activation of PARP by DNA damage is believed to play a role in the cell death consequent to stroke, head trauma, and neurodegenerative diseases. DNA is damaged by excessive amounts of NO produced when the NO synthase enzyme is activated as a result of a series of events initiated by the release of the neurotransmitter glutamate from depolarized nerve terminals (Cosi et al., “Poly(ADP-Ribose) Polymerase Revisited: A New Role for an Old Enzyme: PARP Involvement in Neurodegeneration and PARP Inhibitors as Possible Neuroprotective Agents,” Ann. N.Y. Acad. Sci., 366-379). Cell death is believed to occur as a result of energy depletion as NAD⁺is consumed by the enzyme-catalyzed PARP reaction. Therefore, inhibitors of the PARP enzyme are useful inhibitors of neurotoxicity consequent to stroke, head trauma, and neurodegenerative diseases.

Further, inhibition of PARP should be a useful approach for treatment of conditions or diseases associated with cellular senescence, such as skin aging, through the role of PARP in the signaling of DNA damage. See, e.g., U.S. Pat. No. 5,589,483, which describes a method to extend the lifespan and proliferative capacity of cells comprising administering a therapeutically effective amount of a PARP inhibitor to the cells under conditions such that PARP activity is inhibited. Hence, inhibitors of the PARP enzyme are useful therapeutics for skin aging.

In yet a further application, PARP inhibition is being explored at the clinical level to prevent development of insulin-dependent diabetes mellitus in susceptible individuals (Saldeen et al., “Nicotinamide-induced apoptosis in insulin producing cells in associated with cleavage of poly(ADP-ribose) polymerase,” Mol. Cellular Endocrinol. (1998), 139:99-107). PARP inhibitors should therefore be useful as diabetes-prevention therapeutics.

PARP inhibition is also an approach for treating inflammatory conditions such as arthritis (Szabo et al., “Protective effect of an inhibitor of poly(ADP-ribose) synthetase in collagen-induced arthritis,” Portland Press Proc. (1998), 15:280-281; Szabo, “Role of Poly(ADP-ribose) Synthetase in Inflammation,” Eur. J. Biochem. (1998), 350(1):1-19; Szabo et al., “Protection Against Peroxynitrite-induced Fibroblast Injury and Arthritis Development by Inhibition of Poly(ADP-ribose) Synthetase,” Proc. Natl. Acad. Sci. USA (1998), 95(7):3867-72). PARP inhibitors are therefore useful as therapeutics for inflammatory conditions.

Inhibition of PARP has usefulness for protection against myocardial ischemia and reperfusion injury (Zingarelli et al., “Protection against myocardial ischemia and reperfusion injury by 3-aminobenzamide, an inhibitor of poly(ADP-ribose) synthetase,” Cardiovascular Research (1997), 36:205-215). Therefore, PARP inhibitors are useful in therapy of cardiovascular diseases.

The PARP family of enzymes is extensive. It has recently been shown that tankyrases, which bind to the telomeric protein TRF-1, a negative regulator of telomere length maintenance, have a catalytic domain that is strikingly homologous to PARP and have been shown to have PARP activity in vitro. It has been proposed that telomere function in human cells is regulated by poly(ADP-ribosyl)ation. PARP inhibitors have utility as tools to study this function. Further, as a consequence of regulation of telomerase activity by tankyrase, PARP inhibitors should have utility as agents for regulation of cell life-span, e.g., for use in cancer therapy to shorten the life-span of immortal tumor cells, or as anti-aging therapeutics, since telomere length is believed to be associated with cell senescence.

Competitive inhibitors of PARP are known. For example, Banasik et al. (“Specific Inhibitors of Poly(ADP-Ribose) Synthetase and Mono(ADP-Ribosyl)transferase,” J. Biol. Chem. (1992) 267: 1569-1575) examined the PARP-inhibiting activity of 132 compounds, the most potent of which were 4-amino-1,8-naphthalimide, 6(5H-phenanthridone, 2-nitro-6(5H)-phenanthridone, and 1,5-dihydroxyisoquinoline. Griffin et al. reported the PARP-inhibiting activity for a series of benzamide compounds (U.S. Pat. No. 5,756,510; see also “Novel Potent Inhibitors of the DNA Repair Enzyme poly(ADP-ribose)polymerase (PARP),” Anti-Cancer Drug Design (1995), 10:507-514) and quinalozinone compounds (International Publication No. WO 98/33802). Suto et al. reported PARP inhibition by a series of dihydroisoquinoline compounds (“Dihydroisoquinolines: The Design and Synthesis of a New Series of Potent Inhibitors of Poly(ADP-ribose) Polymerase,” Anti-Cancer Drug Design (1991), 7:107-117). Griffin et al. have reported other PARP inhibitors of the quinazoline class (“Resistance-Modifying Agents. 5. Synthesis and Biological Properties of Quinazoline Inhibitors of the DNA Repair Enzyme Poly(ADP-ribose) Polymerase (PARP),” J. Med. Chem., ASAP Article 10.1021/jm980273t S0022-2623(98)00273-8; Web Release Date: Dec. 1, 1998). Nonetheless, there is still a need for water soluble, small-molecule compounds that are potent PARP inhibitors, especially those that have physical and chemical properties desirable for pharmaceutical applications.

### SUMMARY OF THE INVENTION

The present invention is directed to salts of 8-fluoro-2-(4-methylaminomethyl-phenyl)-1,3,4,5-tetrahydro-azepino[5,4,3-cd]indol-6-one, that function as potent poly(ADP-ribosyl)transferase (PARP) inhibitors, have appreciable water solubilities and are useful as therapeutics, especially in treatment of cancers and the amelioration of the effects of stroke, head trauma, and neurodegenerative disease. As cancer therapeutics, the compounds of the invention may be used in combination with DNA-damaging cytotoxic agents, for example, topotecan, irinotecan, or temozolomide, and/or radiation.

In particular, the present invention is directed to the phosphate salt of 8-fluoro-2-(4-methylaminomethyl-phenyl)-1,3,4,5-tetrahydro-azepino[5,4,3-cd]indol-6-one, having formula (I):

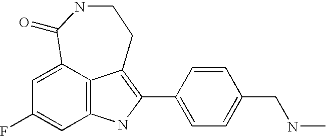

The present invention is also directed to pharmaceutical compositions comprising an effective PARP-inhibiting amount of a phosphate salt of a compound of formula I together with a pharmaceutically acceptable carrier therefor.

The present invention is also directed to a method of inhibiting PARP enzyme activity in vivo, comprising contacting the enzyme with an effective amount of a water soluble salt, preferably a phosphate, salt of a compound of formula (I). These water soluble salts of the invention are potent PARP inhibitors and preferably have a PARP-inhibiting activity corresponding to a Kᵢ of 100 μM or less in the PARP enzyme inhibition assay.

The present invention is further directed to a method of potentiating the cytotoxicity of a cytotoxic drug or ionizing radiation, comprising contacting cells with an effective amount of a water soluble salt preferably a phosphate salt of a compound of formula (I), in combination with a cytotoxic drug or ionizing radiation. The pharmaceutically acceptable salts of the invention preferably have a cytotoxicity potentiation activity corresponding to a PF₅₀ of at least 1 in the cytotoxicity potentiation assay.

The invention also provides therapeutic interventions appropriate in disease or injury states where PARP activity is deleterious to a patient, the therapeutic methods comprising inhibiting PARP enzyme activity in the relevant tissue of the patient by administering a phosphate salt of formula (I). In one such therapeutic intervention method provided by the present invention, the effectiveness of a cytotoxic drug or radiotherapy administered to a mammal in the course of therapeutic treatment is improved by administering to the mammal in need of treatment an effective PARP-inhibiting amount of a phosphate salt of formula (I), in conjunction with the administration of the cytotoxic drug or radiotherapy.

Another therapeutic intervention method provided by the present invention is for delaying the onset of cell senescence associated with skin aging in a mammal, comprising administering to fibroblast cells in the mammal an effective PARP-inhibiting amount of a phosphate salt of formula (I). Yet another therapeutic intervention method provided by the present invention is a method for reducing the neurotoxicity consequent to stroke, head trauma, and neurodegenerative diseases in a mammal by administering an effective amount of a phosphate salt of formula (I), to the mammal.

The compounds of the present invention provide a therapeutic approach to treatment of inflammatory conditions, comprising administering an effective amount of a phosphate salt of formula (I), to a mammal in need of treatment.

Yet a further therapeutic intervention method provided by the present invention is a cardiovascular therapeutic method for protecting against myocardial ischemia and reperfusion injury in a mammal, comprising administering to the mammal an effective amount of a phosphate salt of formula (I).

### DETAILED DESCRIPTION OF THE INVENTION AND PREFERRED EMBODIMENTS

PARP-Inhibiting Agents:

The synthesis of 8-fluoro-2-(4-methylaminomethyl-phenyl)-1,3,4,5-tetrahydro-azepino[5,4,3-cd]indol-6-one, was described in U.S. Pat. No. 6,495,541 herein incorporated by reference. As used herein, the terms “comprising” and “including” are used herein in their open, non-limiting sense.

The term “halogen” represents chlorine, fluorine, bromine or iodine. The term “halo” represents chloro, fluoro, bromo or iodo.

In the case of compounds, salts, or solvates that are solids, it is understood by those skilled in the art that the inventive compounds, salts, and solvates may exist in different crystalline or polymorph forms, all of which are intended to be within the scope of the present invention and specified formulas.

In some cases, the inventive compounds will have chiral centers. When chiral centers are present, the inventive compounds may exist as single stereoisomers, racemates, and/or mixtures of enantiomers and/or diastereomers. All such single stereoisomers, racemates, and mixtures thereof are intended to be within the broad scope of the generic structural formulae (unless otherwise indicated). Preferably, however, the inventive compounds are used in essentially optically pure form (as generally understood by those skilled in the art, an optically pure compound is one that is enantiomerically pure). Preferably, the compounds of the invention are at least 90% of the desired single isomer (80% enantiomeric excess), more preferably at least 95% (90% e.e.), even more preferably at least 97.5% (95% e.e.), and most preferably at least 99% (98% e.e.).

In some cases, compounds can occur in tautomeric forms. In such cases, it is intended that both tautomers are encompassed by the structural formulae.

Pharmaceutical Methods and Compositions:

The invention is also directed to a method of inhibiting PARP enzyme activity, comprising contacting the enzyme with an effective amount of a water soluble salt of formula (I), for example a phosphate salt of formula (I), or a solvate of the water soluble salt thereof. For example, PARP activity may be inhibited in mammalian tissue by administering a water soluble salt of formula (I) for example, a phosphate salt, or solvate of said salts thereof.

“Treating” or “treatment” is intended to mean mitigating or alleviating an injury or a disease condition in a mammal, such as a human (e.g., a patient), that is mediated by the inhibition of PARP activity, such as by potentiation of anti-cancer therapies or inhibition of neurotoxicity consequent to stroke, head trauma, and neurodegenerative diseases. Types of treatment include: (a) as a prophylactic use in a mammal, particularly when the mammal is found to be predisposed to having the disease condition but not yet diagnosed as having it; (b) inhibition of the disease condition; and/or (c) alleviation, in whole or in part, of the disease condition.

One treatment method involves improving the effectiveness of a cytotoxic drug or radiotherapy administered to a mammal in the course of therapeutic treatment, comprising administering to the mammal an effective amount of a phosphate salts of formula 1 in conjunction with administration of the cytotoxic drug (e.g., topotecan or irinotecan) or radiotherapy. The PARP-inhibiting phosphate salts of formula 1 may also be advantageously used in a method for reducing neurotoxicity consequent to stroke, head trauma, and neurodegenerative diseases in a mammal by administering a therapeutically effective amount of phosphate salts of formula 1 to the mammal. The PARP-inhibiting salts of the invention may also be used in a method for delaying the onset of cell senescence associated with skin aging in a human, comprising administering to fibroblast cells in the human an effective PARP-inhibiting amount of the phosphate salts of formula 1. Further, the phosphate salts of formula 1 may also be used in a method for helping prevent the development of insulin-dependent diabetes mellitus in a susceptible individual, comprising administering a therapeutically effective amount of the salt. Additionally, the phosphate salts of formula 1 may also be employed in a method for treating an inflammatory condition in a mammal, comprising administering a therapeutically effective amount of the salt to the mammal. Moreover, the agents may also be used in a method for treating cardiovascular disease in a mammal, comprising administering to the mammal a therapeutically effective amount of a PARP-inhibiting of a phosphate salt of formula 1. As knowledge regarding the therapeutic roles of PARP inhibitors progresses in the art, other utilities of the PARP-inhibiting salts of the invention will become apparent.

The activity of the inventive compounds as inhibitors of PARP activity may be measured by any of the suitable methods known or available in the art, including by in vivo and in vitro assays. An example of a suitable assay for activity measurements is the PARP enzyme inhibition assay described in U.S. Pat. No. 6,495,541 herein incorporated by reference in its entirety for all purposes.

Administration of the phosphate or glucuronate salts of formula 1 may be performed according to any of the accepted modes of administration available in the art. Illustrative examples of suitable modes of administration include oral, nasal, parenteral, topical, transdermal, intravenous and rectal delivery. Oral and intravenous delivery are preferred routes of administration.

The phosphate salts of formula (I), or a pharmaceutically acceptable or solvate thereof may be administered as a pharmaceutical composition in any pharmaceutical form recognizable to the skilled artisan as being suitable. Suitable pharmaceutical forms include solid, semisolid, liquid, or lyophilized formulations, such as tablets, powders, capsules, suppositories, suspensions, liposomes, and aerosols. Pharmaceutical compositions of the invention may also include suitable excipients, diluents, vehicles, and carriers, as well as other pharmaceutically active agents (including other PARP-inhibiting agents), depending upon the intended use.

Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate to give the desired products for oral, parenteral, topical, intravaginal, intranasal, intrabronchial, intraocular, intraaural, and/or rectal administration.

Solid or liquid pharmaceutically acceptable carriers, diluents, vehicles, or excipients may be employed in the pharmaceutical compositions. Illustrative solid carriers include starch, lactose, calcium sulphate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition contains at least a therapeutically effective amount of a PARP-inhibiting agent (i.e., a phosphate salt of formula (I), or a solvate thereof), and preferably contains one or more pharmaceutical dosage units. The selected dose may be administered to a mammal, for example, a human patient, in need of treatment of a condition mediated by inhibition of PARP activity, by any known or suitable method of administering the dose, including: topically, for example, as an ointment or cream; orally; rectally, for example, as a suppository; parenterally by injection; or continuously by intravaginal, intranasal, intrabronchial, intraaural, or intraocular infusion. A “therapeutically effective amount” is intended to mean the amount of an agent that, when administered to a mammal in need thereof, is sufficient to effect treatment for injury or disease condition mediated by inhibition of PARP activity, such as for potentiation of anti-cancer therapies and inhibition of neurotoxicity consequent to stroke, head trauma, and neurodegenerative diseases. The amount of a given compound of the invention that will be therapeutically effective will vary depending upon factors such as the particular compound, the disease condition and the severity thereof, the identity of the mammal in need thereof, which amount may be routinely determined by artisans.

It will be appreciated that the actual dosages of the PARP-inhibiting agents used in the pharmaceutical compositions of this invention will be selected according to the particular complex being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.001 to about 1000 mg/kg body weight, with courses of treatment repeated at appropriate intervals.

Synthetic Processes:

The present invention is further directed to methods of synthesizing the PARP-inhibiting agents by processes such as those set forth below for exemplary compounds of the invention. In the following examples, the structures of the compounds were confirmed by one or more of the following: proton magnetic resonance spectroscopy, infrared spectroscopy, elemental microanalysis, mass spectrometry, thin layer chromatography, high performance liquid chromatography, and melting point.

Elemental microanalyses were performed by Atlantic Microlab Inc. (Norcross, Ga.) or Galbraith Laboratories (Nashville, Tenn.), and gave results for the elements stated within ±0.4% of the theoretical values. Flash column chromatography was performed using Silica gel 60 (Merck Art 9385). Analytical thin layer chromatography (TLC) was performed using precoated sheets of Silica 60 F₂₅₄ (Merck Art 5719). Melting points (mp) were determined on a MelTemp apparatus and are uncorrected. All reactions were performed in septum-sealed flasks under a slight positive pressure of argon, unless otherwise noted. All commercial solvents were reagent-grade or better and used as supplied.

The following abbreviations may be used herein: Et₂O (diethyl ether); DMF (N,N-dimethylformamide); DMSO (dimethylsulfoxide); MeOH (methanol); EtOH (ethanol); EtOAc (ethyl acetate); THF (tetrahydrofuran); Ac (acetyl); Me (methyl); Et (ethyl); and Ph (phenyl). The general reaction protocols described below may be used to prepare the compounds of the invention and assay the water solubility of the salts.

Water Solubilities of Different Salt Forms

| Salt Form of Formula 1 | Solubility (mg/mL) |
|---|---|
| Free base | 0.18 |
| Hydrochloride | 1.6 |
| Mesylate | 15.5 |
| Gluconate | >128 |
| Tartrate | 1.1 |
| Acetate | 8.8 |
| Glucuronate | 89 |
| Phosphate | 2.8 |

Water Solubility Assay

Weigh approximately 1.0 mg of 8-fluoro-2-(4-methylaminomethyl-phenyl)-1,3,4,5-tetrahydro-azepino[5,4,3-cd]indol-6-one (free base) into a scintillation vial, and then add 2.0 ml Milli Q water. The sample suspension was stirred at room temperature for 3 hours. The suspension was transferred into an eppendorf vial and centrifuged at 14000 rpm for 8 minutes. The supernant solution was then assayed by HPLC.

Weigh approximately 5.0 mg 8-fluoro-2-(4-methylaminomethyl-phenyl)-1,3,4,5-tetrahydro-azepino[5,4,3-cd]indol-6-one phosphate or any other salt of formula 1 into a scintillation vial, and then add 1.0 ml Milli Q water. The suspension was stirred at room temperature for 3 hours and then centrifuged at 14000/min for 8 minutes. The supernant was diluted 10 times by Milli Q water. The final solution was then assayed by HPLC.

Standard Preparation:

Accurately weigh out 2.5-3.0 mg of AG014447 reference standard into 10 ml volumetric flask, and then bring to volume with MeOH. Mix thoroughly.

HPLC Conditions:

Buffer: 25 mM ammonium phosphate buffer (pH 2.5)

Organic Modifier: Acetonitrile (ACN)

Wavelength; 210 nm

Column: Waters Symmetry C18, 4.6×150 mm, 5 μm;

Flow rate: 1.0 mL/minute

Injection Volume: 5 μL

Run time: 24 minutes

Column Temp: Ambient

Gradient:

| Time | % Buffer | ACN (%) |
|---|---|---|
| 0 | 90 | 10 |
| 15 | 60 | 40 |
| 20 | 60 | 40 |
| 20.1 | 90 | 10 |
| 24 | 90 | 10 |

Calculations:

The solubility of the sample is calculated by the equation below:

S=A/As×Cs×D

Where A is the peak area of the sample; As is the peak area of the standard; Cs is the concentration of the standard solution; D is the dilution factor.

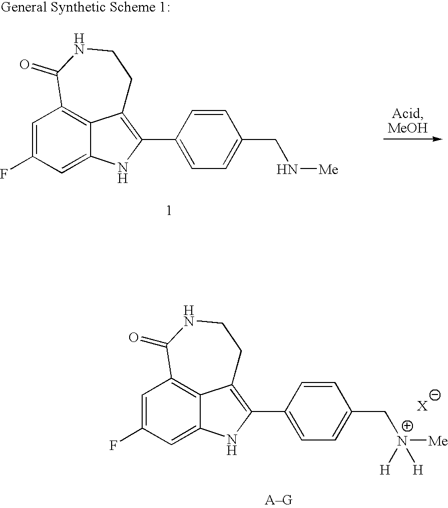

In Scheme 1, the amine 1 was treated with various acids in methanol. The resulting salt was lyophilized and further purified by recrystallization, if necessary.

### EXAMPLES

### Example A

### 8-Fluoro-2-(4-methylaminomethyl-phenyl)-1,3,4,5-tetrahydro-azepino[5,4,3-cd]indol-6-one Mesylate

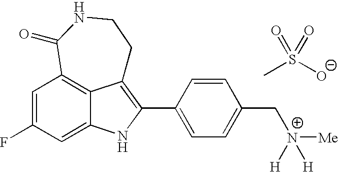

8-Fluoro-2-(4-methylaminomethyl-phenyl)-1,3,4,5-tetrahydro-azepino[5,4,3-cd]indol-6-one (259 mg, 0.801 mmol) was partially dissolved in methanol (5 mL) and was then treated with methane sulfonic acid (1.0 M methanolic solution, 0.801 mL). The amine was completely dissolved by gently heating the solution and by using an additional amount of methanol (10 mL). The solution was filter through cotton to separate any particulates. The solution was partially concentrated down in vacuo. 1 mL of deionized water was added and the methanol was completely evaporated in vacuo. The product was lyophilized to give 326 mg (97%) as a bright yellow solid: Anal. (C₂₀H₂₂FN₃O₄ 2H₂O)C, H, N.

### Example B

### 8-Fluoro-2-(4-methylaminomethyl-phenyl)-1,3,4,5-tetrahydro-azepino[5,4,3-cd]indol-6-one Hydrochloride

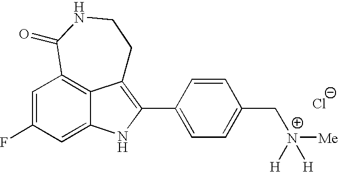

In a manner similar to that described for Example A, 8-fluoro-2-(4-methylaminomethyl-phenyl)-1,3,4,5-tetrahydro-azepino[5,4,3-cd]indol-6-one (30 mg, 0.093 mmol), and HCl (0.10 M aqueous solution, 0.90 mL) were used to yield 8-fluoro-2-(4-methylaminomethyl-phenyl)-1,3,4,5-tetrahydro-azepino[5,4,3-cd]indol-6-one hydrochloride, 33 mg (99%) as a bright yellow solid: Anal. (C₁₉H₁₉FN₃OCl 0.3H₂O)C, H, N.

### Example C

### 8-fluoro-2-(4-methylaminomethyl-phenyl)-1,3,4,5-tetrahydro-azepino[5,4,3-cd]indol-6-one Acetate

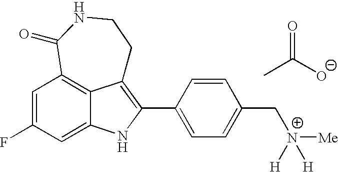

In a manner similar to that described for Example A, 8-fluoro-2-(4-methylaminomethyl-phenyl)-1,3,4,5-tetrahydro-azepino[5,4,3-cd]indol-6-one (30.8 mg, 0.0952 mmol), and acetic acid (1.0 M methanolic solution, 0.952 mL) were used to yield 8-fluoro-2-(4-methylaminomethyl-phenyl)-1,3,4,5-tetrahydro-azepino[5,4,3-cd]indol-6-one acetate, 36.1 mg (99%) as a bright yellow solid: Anal. (C₂₁H₂₂FN₃O₃ 1.5H₂O)C, H, N.

### Example D

### 8-Fluoro-2-(4-methylaminomethyl-phenyl)-1,3,4,5-tetrahydro-azepino[5,4,3-cd]indol-6-one Gluconate

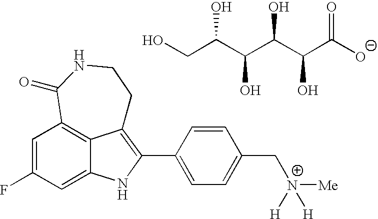

In a manner similar to that described for Example A, 8-fluoro-2-(4-methylaminomethyl-phenyl)-1,3,4,5-tetrahydro-azepino[5,4,3-cd]indol-6-one (30.2 mg, 0.0934 mmol), and gluconic acid (2.55 M aqueous solution, 0.0366 mL) were used to yield 8-fluoro-2-(4-methylaminomethyl-phenyl)-1,3,4,5-tetrahydro-azepino[5,4,3-cd]indol-6-one gluconate, 47.5 mg (98%) as a bright yellow solid: Anal. (C₂₅H₃₀FN₃O₈ 1.9H₂O)C, H, N.

### Example E

### 8-Fluoro-2-(4-methylaminomethyl-phenyl)-1,3,4,5-tetrahydro-azepino[5,4,3-cd]indol-6-one Tartrate

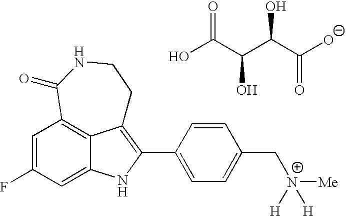

In a manner similar to that described for Example A, 8-fluoro-2-(4-methylaminomethyl-phenyl)-1,3,4,5-tetrahydro-azepino[5,4,3-cd]indol-6-one (30.0 mg, 0.0928 mmol), and L-tartaric acid (1.0 M methanolic solution, 0.0928 mL) were used to yield 8-fluoro-2-(4-methylaminomethyl-phenyl)-1,3,4,5-tetrahydro-azepino[5,4,3-cd]indol-6-one tartrate, 42.7 mg (97%) as a bright yellow solid: Anal. (C₂₃H₂₄FN₃O₇ 1.8H₂O)C, H, N.

### Example F

### 8-Fluoro-2-(4-methylaminomethyl-phenyl)-1,3,4,5-tetrahydro-azepino[5,4,3-cd]indol-6-one Glucuronate

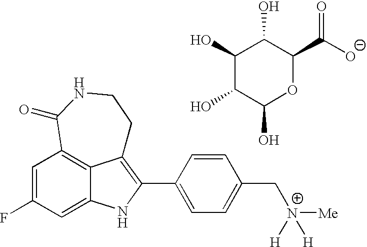

In a manner similar to that described for Example A, 8-fluoro-2-(4-methylaminomethyl-phenyl)-1,3,4,5-tetrahydro-azepino[5,4,3-cd]indol-6-one (30.0 mg, 0.0928 mmol), and glucoronic acid (0.5 M aqueous solution, 0.186 mL) were used to yield 8-fluoro-2-(4-methylaminomethyl-phenyl)-1,3,4,5-tetrahydro-azepino[5,4,3-cd]indol-6-one glucuronate, 47.9 mg (100%) as a bright yellow solid: Anal. (C₂₅H₂₈FN₃O₈ 1.9H₂O)C, H, N.

### Example G

### 8-Fluoro-2-(4-methylaminomethyl-phenyl)-1,3,4,5-tetrahydro-azepino[5,4,3-cd]indol-6-one Phosphate

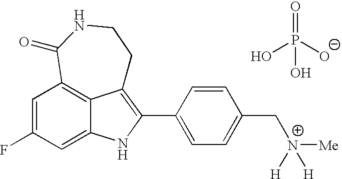

In a manner similar to that described for Example A, 8-fluoro-2-(4-methylaminomethyl-phenyl)-1,3,4,5-tetrahydro-azepino[5,4,3-cd]indol-6-one (42.0 mg, 0.130 mmol), and phosphoric acid (0.5 M aqueous solution, 0.260 mL) were used to yield 8-fluoro-2-(4-methylaminomethyl-phenyl)-1,3,4,5-tetrahydro-azepino[5,4,3-cd]indol-6-one phosphate after lyophilization and recrystallization in 0.5: 6.5: 3H₂O: methanol: CH₂Cl₂ to give 32.2 mg (58%) as a bright yellow solid: Anal. (C₁₉H₂₁FN₃O₅P 1.9H₂O)C, H, N.

PARP Enzyme Inhibition Assay:

The PARP enzyme-inhibiting activities of the compounds of the invention were assayed as described by Simonin et al. (J. Biol. Chem. (1993), 268:8529-8535) and Marsischky et al. (J. Biol. Chem. (1995), 270:3247-3254) with minor modifications as follows. Samples (50 μL) containing 20 nM purified PARP protein, 10 μg/mL DNAse I-activated calf thymus DNA (sigma), 500 μM NAD⁺, 0.5 μCi [³²P]NAD⁺, 2% DMSO, and various concentrations of test compounds were incubated in sample buffer (50 mM Tris pH 8.0, 10 mM MgCl₂, 1 mM tris(carboxyethyl)phosphine HCl) at 25° C. for 5 minutes. Under these conditions, the reaction rate was linear for times up to 10 minutes. The reaction was stopped by the addition of an equal volume of ice-cold 40% trichloroacetic acid to the samples, which were then incubated on ice for 15 minutes. The samples were then transferred to a Bio-Dot microfiltration apparatus (BiORad), filtered through Whatman GF/C glass-fiber filter paper, washed 3 times with 150 μL of wash buffer (5% trichloroacetic acid, 1% inorganic pyrophosphate), and dried. [³²P]ADP-Ribose incorporation into the acid-insoluble material was quantitated using a PhosphorImager (Molecular Dynamics) and ImageQuant software. Inhibition constants (Kᵢ) were calculated by non-linear regression analyses using the velocity equation for competitive inhibition (Segel, Enzyme Kinetics: Behavior and Analysis of Rapid Equilibrium and Steady-State Enzyme Systems, John Wiley & Sons, Inc., New York (1975), 100-125). In the case of tight-binding inhibitors, 5 nM enzyme was used and the reaction was incubated at 25° C. for 25 minutes. Kᵢ values for tight-binding inhibitors were calculated using the equation described by Sculley et al. (Biochim. Biophys. Acta (1986), 874:44-53).

Cytotoxicity Potentiation Assay:

A549 cells (ATCC, Rockville, Md.) were seeded into 96-well cell culture plates (Falcon brand, Fisher Scientific, Pittsburgh, Pa.) 16 to 24 hours before experimental manipulation. Cells were then treated with a test compound (or a combination of test compounds where indicated) for either 3 days or 5 days, at a concentration of 0.4 μm. At the end of treatments, relative cell number was determined either by MTT assay or SRB assay. For the MTT assay, 0.2 μg/μl of MTT (3-(4,5-dimethylthiazol-2-yl)-2,5-diphenyltetrazolium bromide, Sigma Chemical Co., St. Louis, Mo.) was added to each well of a plate, and the plate was incubated in a cell-culture incubator for 4 hours. Metabolized MTT in each well was solubilized in 150 μl of DMSO (Sigma Chemical Co.) with shaking and quantified with a Wallac 1420 Victor plate reader (EG&G Wallac, Gaithersburg, Md.) at 540 nm. For the SRB assay, cells were fixed with 10% trichloroacetic acid (Sigma Chemical Co) for an hour at 4° C. After extensively washing, fixed cells were stained for 30 minutes with 0.4% sulforhodamine B (SRB, Sigma Chemical Co.) in 1% acetic acid (Sigma Chemical Co). Unbound SRB was washed away with 1% acetic acid. Then the cultures were air-dried, and bound dye was solubilized with 10 mM unbuffered Tris base (Sigma Chemical Co) with shaking. The bound dye was measured photometrically with the Wallac Victor plate reader at 515 nm. The ratio of the OD (optical density) value of a compound-treated culture to the OD value of a mock-treated culture, expressed in percentage, was used to quantify the cytotoxicity of a compound. The concentration at which a compound causes 50% cytotoxicity is referred to as IC₅₀. To quantify the potentiation of the cytotoxicity of topotecan or temozolomide by test compounds, a dimensionless parameter PF₅₀ is used and is defined as the ratio of the IC₅₀ of topotecan or temozolomide alone to the IC₅₀ of topotecan or temozolomide in combination with a test compound. For the compounds of the invention, PF₅₀ values were determined by testing with topotecan.

Inhibition constants (Kᵢ values) and cytotoxicity potentiation parameters (PF₅₀ values) as determined for exemplary compounds of the invention are presented in Table 1 below. If there are two Kᵢ values for a single compound, it means that the compound Kᵢ was tested twice.

TABLE 1
PARP Enzyme Inhibition and Cytotoxicity Potentiation

| | Inhibition Constant | Cytotoxicity Potentiation |
|---|---|---|
| Compound No. | Kᵢ (nM) | PF₅₀ |
| Formula 1, free base | 4.4 | 2.4 |

While the invention has been described by reference to preferred embodiments and specific examples, those skilled in the art will recognize that various changes and modifications can be made without departing from the spirit and scope of the invention. Thus, the invention should be understood as not being limited by the foregoing detailed description, but as being defined by the appended claims and their equivalents.

All U.S. and foreign patents, published patent applications, and other references cited herein are hereby incorporated by reference in their entireties.

What is claimed:

1. A phosphate salt of 8-fluoro-2-(4-methylaminomethyl-phenyl)-1,3,4,5-tetrahydro-azepino[5,4,3-cd]indol-6-one.

2. A pharmaceutical composition of the compound of claim 1, suitable for oral administration comprising a pharmaceutically effective dose of the compound of claim 1 and a pharmaceutically acceptable carrier thereof.

3. A pharmaceutical composition of the compound of claim 1, suitable for injectable administration comprising a pharmaceutically effective dose of the compound of claim 1 and a pharmaceutically acceptable carrier thereof.

4. A chemotherapy combination a pharmaceutically effective dose of 8-fluoro-2-(4-methylaminomethyl-phenyl)-1,3,4,5-tetrahydro-azepino[5,4,3-cd]indol-6-one phosphate and a chemotherapeutic agent selected from irenotecan, temozolamide and dacarbazine.

5. The chemotherapeutic combination of claim 4 wherein the chemotherapeutic agent is irenotecan.

6. The chemotherapeutic combination of claim 4 wherein the chemotherapeutic agent is temozolamide.

7. The chemotherapeutic combination of claim 4 wherein the chemotherapeutic agent is dacarbazine.

8. A method of improving the effectiveness of a cytotoxic drug or radiotherapy administered to a mammal in the course of therapeutic treatment, said method comprising: administering to the mammal an effective PARP-inhibiting amount of the compound of claim in conjunction with the administration of said cytotoxic drug or radiotherapy.

9. A method for protecting against injury consequent to myocardial ischemia or reperfusion in a mammal comprising: administering to the mammal an effective amount of the compound, defined in claim 1.

10. A method for reducing neurotoxicity consequent to a stroke, a head trauma, or a neurodegenerative disease in a mammal comprising: administering to the mammal an effective amount of the compound defined in claim 1.

11. A method for delaying the onset of cell senescence associated with skin aging in a mammal comprising: administering to fibroblast cells in the mammal an effective PARP-inhibiting amount of the compound defined in claim 1.

12. A method for preventing the onset of insulin-dependent diabetes in a mammal comprising administering the compound defined in claim 1 to said mammal.
